(19) United States
(12) Patent Application Publication
(10) Pub. No.: US 2020/0203018 A1
(43) Pub. Date: Jun. 25, 2020

(54) SYSTEM AND METHOD FOR MANAGING HEALTH RISKS RELATING TO IN VIVO INSULIN AND/OR GLUCOSE LEVELS

(71) Applicant: Alon Walter, Givataim (IL)

(72) Inventor: Alon Walter, Givataim (IL)

(21) Appl. No.: 16/676,491

(22) Filed: Nov. 7, 2019

Related U.S. Application Data
(60) Provisional application No. 62/781,621, filed on Dec. 19, 2018.

(51) Int. Cl. G16H 50/30; G16H 20/60; G16H 50/50

(52) U.S. Cl. CPC G16H 50/30; A61B 5/14532; G16H 50/50; G16H 20/60

(57) ABSTRACT

The subject matter discloses a method, the method comprises: calculating score of risk for blood toxicity over a time period, wherein the toxicity is associated with a level of insulin or a level of glucose in blood, wherein input of the calculating includes a dataset of a plurality of data points, each data point includes a first value of the level of glucose or a second value of the level of insulin in a certain time interval within the time period. In one embodiment, each of the data points is received from a sensor, the sensor sampling a user. In one embodiment, each of the data points is received from a processor, the processor simulating the dataset in accordance with behavior of the user.

If the score of risk exceeding a threshold, then it is presented on a computing device. In some embodiment an alert is initiated.

### FIELD OF THE INVENTION

The present disclosure relates to medical detection and analysis in general, and to monitoring blood glucose and insulin levels, in particular.

### BACKGROUND OF THE INVENTION

The effects of insulin and blood glucose levels are of great interest, as insulin resistance and type 2 diabetes have reached epidemic proportions.

For pre-diabetics, solutions revolve around changing and planning their nutrition and their physical activity routines, supervised by periodic assessments of their condition by professionals.

This strategy targets the lifestyle elements that are correlated with the development of insulin resistance. These solutions target the root cause of the problem. Such a solution is considered as a preventative measure, and also as a way to reduce and even fix the underlying problem that led to the pre-diabetic condition (by way of lowering the insulin resistance to a healthy range).

For diabetics, in addition to the former, solutions are focused around medically approved devices that provide accurate measurements of their blood glucose levels, and methods of administrating medications (with the purpose of controlling blood glucose levels) that are guided by the devices that measure the blood glucose levels.

When trying to evaluate the harmful effects of substances (such as glucose and insulin in this case), the most prominent applicable frameworks are: Toxicity Risk Analysis (TRA) and Toxicokinetic-Toxicodynamic (TKTD) models.

Though both frameworks aim to quantify the harmful effects of substances in the form of a risk measure, the TRA approach is very “high level” and “of a posteriori nature”—dealing with statistics regarding symptoms in large populations, while the TKTD approach is very “low level” and “of a priori nature”—simulating biochemical interactions, and deriving the relevant measures as they emerge from the simulations.

### SUMMARY OF THE INVENTION

The term computing device refers herein to a device that includes a processing unit. Examples for such device are a personal computer, a laptop, a server, a tablet, a cellular device and an IOT (internet of things) device.

The term risk value refers to a value that is assigned to a given time interval. This value reflects the amount of risk that is associated with given parameter values that are associated with that time interval. Examples of such parameters are “blood glucose level”, “blood insulin level” and “time of day”.

The term Context of Risk refers to a frame of reference in which risk values are assigned to blood glucose levels or to insulin levels, where the risk is in relation to a specific health issue. Examples of such health issues are “the risk for damage to β-cells” and “the risk for raising the inflammatory state”.

For example, healthy users that want to monitor their risk of developing insulin resistance may select a Context of Risk that is focused on this issue, while a user that has pronounced insulin resistance might want to monitor the risk of developing β-cell dysfunction, and may select such a Context of Risk. In other words, Context of Risk reflects a subject of concern—that relates to the need for monitoring the user's insulin and/or blood glucose levels.

The term dataset refers to a collection of data points included within a certain time period. Each data point includes a value of blood glucose level of the user and/or a value of insulin level of the user, and its associated time interval. The collection may represent a continuous time span, for example one day.

The term Meal Plan refers to a schedule of meals which reflects what the user actually eats. The meal plan is a list of records. Each record contains a food item with some of its nutritional values, and the time and date at which the food item was (or is) planned to be eaten.

The term Meal Plan Template refers to a template that may be used by the user for planning future meals. The Meal Plan Template may include a list of records. Each record may include a food item with some of its nutritional values, and the time of day at which each food item is intended to be eaten. The record does not include the date in which the item is planned to be eaten. The user can plan future meals by assigning a Meal Plan Template to a future date, at which point the user has created a Meal Plan for that date.

The term Score of Risk refers to the value that is calculated based on a dataset of insulin and/or blood glucose levels. The value that is calculated reflects the accumulated risk that is associated with the toxicity of the given insulin and/or the blood glucose levels over the time span of the dataset, and as relevant to the selected Context of Risk.

The term Toxicological Risk Analysis (TRA) refers herein to a process of hazard identification, dose-response assessment and exposure assessment, providing a method to calculate an estimation of the incidence and the severity of adverse effects likely to occur in a human population in relation to actual or eventual exposure to hazardous compounds.

The term Risk Model is borrowed from the realm of TRA (or more generally from the realm of Risk Analysis). The term Risk Model refers to the mathematical representation of the risk that is estimated for each concentration of a toxin.

The term B_G Basal refers herein to an estimation of the fasting blood glucose level (FBG) of the user.

One exemplary embodiment of the disclosed subject matter is a system and method for monitoring blood glucose levels. Such a system provides proper risk assessment and management regarding health implications associated with glucose and insulin levels.

According to some embodiments, the system periodically calculates the Score of Risk.

According to some embodiments, the Score of Risk is a time integration of the risk values assigned to data points within a given dataset. The risk value of a data point is calculated by a Risk Function. The Risk Function is selected or generated according to the Context of Risk of the user. The Context of Risk may be selected by the user or may be dynamically defined according to the history of the user and/or according to the profile of the user.

According to some embodiments, the Score of Risk is presented to the user. The Score of Risk represents the cumulative health risk (accumulated exposure to toxicity) attributed to the user's insulin and/or blood glucose levels (as outlined by the Context of Risk) in the time span corresponding to the dataset.

According to some embodiments, the blood glucose levels are obtained from measurements received by measuring devices such as a Continuous Glucose Monitor (CGM) and a blood glucose meter (BGM). The CGM may measure blood glucose levels directly or indirectly by measuring body fluids such as interstitial fluid, sweat or tears, or by other invasive or noninvasive means, such as optical or electrical sensors. The CGM or BGM device may be a wearable device. The measurements may be also obtained by finger-prick tests.

The blood glucose/insulin levels may also be obtained from calculations, for example by deriving the blood glucose levels from a Meal Plan and the user's reaction to sugar according to a standard glucose tolerance test. Examples of such calculations are:

A mathematical time step simulation that uses nutritional-intake-records to propagate the state of the user's modeled compartments, such as described in the paper: Meal simulation model of the glucose-insulin system. Dalla Man C, Rizza R A, Cobelli C. (IEEE Trans Biomed Eng. 2007 October;54(10):1740-9.) and as was implemented in Simulink, as described in: https://www.mathworks.com/help/simbio/examples/simulating-the-glucose-insulin-response.html.

A convolution of the user's blood glucose response with the carbohydrate intake over time (e.g. as can be directly derived from the user's nutritional-intake-records).

The blood glucose levels may also be obtained from a combination of measurements and calculations. Examples of blood glucose levels obtained from a combination of measurements and calculations:

A mathematical simulation that uses blood glucose measurements to adapt the user's profile-derived-parameters, such that the simulated values fit better with the measured values.

A health data aggregate receives an intermittent sequence of blood glucose level measurements and extrapolates or estimates the blood glucose levels for an unsampled time period before, within, or after the sequence.

A source for datasets may also be the result of data processing of other datasets. Examples of sources for datasets originating as a result of data processing of other datasets:

A health data aggregate or an application that receives a dataset comprised of raw data measurements, and performs data filtering, as to smooth or estimate better values from multiple measurements for each data point in the resulting dataset.

Calculating standardized samples, for example, by a health data aggregate or an application that receives a dataset with inconsistent time sampling intervals, and performs resampling (e.g. via spline interpolation), resulting with a dataset that has values with a regular time interval.

According to some embodiments, prior to calculating a Score of Risk, the system identifies the Context of Risk from previously measured blood glucose levels. The Context of Risk may be, for example, “the risk for damage to β-cells”, “the risk for raising the inflammatory state”, etc.

According to some embodiments, the Context of Risk is predefined by the system or is selected by the user. In some embodiments the Context of Risk is adjusted by the system as the user's condition evolves. For example: a healthy user may choose the Context of Risk to be “risk of gaining weight”. However, if the system identifies that the measured blood glucose levels are above the expected range of a healthy person, the system may set the Context of Risk to “risk for damage to β-cells”. The method for adjusting the Context of Risk is explained in greater detail in FIG. 5.

Risk Assignment:

According to some embodiments, risk is assigned by implementation of a specialized risk analysis approach, tailored to the idiosyncrasies of the problem in hand, in a manner that can be seen as a combination of TRA (statistical) and TKTD (analytical simulation). This risk analysis approach adapts the concept of a Risk Model from TRA—in the form of a Risk Function, while determining exposure to glucose and/or insulin by implementing a TKTD-analogous simulation of the glucose-insulin system.

According to some embodiments, the Risk Function assigns a weight factor for each given blood glucose level, in the form:

Factor_{[dimensionless]}=Risk_Function(Blood_Glucose_Level_{[mg/dL]})

or

Factor_{[dimensionless]}=Risk_Function(Blood_Glucose_Level_{[mmol/L]})

The Risk Function correlates between a given blood glucose concentration and its associated risk-weight-factor, expressing instantaneous damage as arising both directly from the implied glucose toxicity, and indirectly, due to other sources that are correlated with the high levels of hyperglycemia. The same structure and correlation to risk are relevant when considering the Risk Function for blood insulin levels.

According to some embodiments, the Risk Function is built based on the Risk Model of the current Context of Risk. The TRA methodology for determining the Risk Model is first to identify which of the 4 basic model-types is appropriate, and then use available toxicological research data to determine the parameters of the identified model-type, such that the function generated based on these parameters best fits the available research data. That generated function is the Risk Model.

According to some embodiments, the Risk Model determination-process assumes that for normal concentration levels in healthy individuals, glucose and insulin are not considered toxic; This descriptive constraint effectively disqualifies model-types that inherently have positive risk values for every non-zero concentration, and hence establishes that the model-type of the Risk Model is either a Threshold-model-type or a Hormesis-model-type.

According to some embodiments, the Risk Model is based on the Threshold-model-type. The Threshold model has a (namesake) threshold-concentration value, for which it is known (or assumed) that for concentrations bellow it, the risk values are zero. For simplicity, the behavior above the threshold-concentration is assumed to increase linearly, such that it emerges from the threshold-concentration (i.e., the indicated emerging line crosses the horizontal concentration axis at the threshold-concentration).

According to some embodiments, the Risk Model requires two values to be completely defined:

1. The threshold-concentration.
2. The slope for the linear component above the threshold-concentration.

When the risk assessment is contextual, these values are derived based on the Context of Risk. If the Context of Risk is not known, first it should be determined either by the user's selection or by the system.

For the general case:

$$Risk\_Model ( BG\_Level ) = \{ \begin{matrix}0 , & BG\_Level < {TH}_{1} \\ {F}_{0} \cdot ( BG\_Level - {TH}_{1} ) , & BG\_Level \geq {TH}_{1}\end{matrix}$$

The Risk Function based on the Risk Model:

$$Risk\_Function ( BG\_Level ) = \{ \begin{matrix}0 , & BG\_Level < {TH}_{1} \\ {F}_{1} \cdot ( BG\_Level - {TH}_{1} ) , & BG\_Level \geq {TH}_{1}\end{matrix}$$

Where:

BG_Level is a blood glucose level. Units: [mg/dl] or [mmol/l].

TH₁ is the Threshold-concentration. Units: [mg/dl] or [mmol/l].

F₀ is a factor that defines the Risk Model's slope. Units: [dl/mg] or [l/mmol].

F₁ is a factor that defines the Risk Function's slope. Units: [dl/mg] or [l/mmol].

A point of notice is that the Risk Model's result is dimensionless. In the same manner the Risk Function's result is also dimensionless.

For the exemplary Context of Risk of “β-cell dysfunction”, TH₁=150 [mg/dl] (˜8.3 [mmol/l]).

The value of F₁ can be expressed as (defined in the description of block 410):

$${F}_{1} = \frac{{y}_{2} - {y}_{1}}{{x}_{2} - {x}_{1}} = \frac{20}{BG\_H - {TH}_{1}}$$

For a typical user applicable to this exemplary Context of Risk, a high blood glucose level (denoted as BG_H) is 250 [mg/dl] (˜13.9 [mmol/l]):

$${F}_{1} = \frac{20}{BG\_H - {TH}_{1}} = \frac{20}{{100}_{[ mg / dL ]}} = {0.2}_{[ dL / mg ]} = {9}_{[ l / mmol ]}$$

And thus, the implementation of the Risk Function for the current Context of Risk is explicitly expressed as:

$$Risk\_Function ( BG\_Level ) = \{ \begin{matrix}0 , & BG\_Level < 150 \\ 0.2 \cdot ( BG\_Level - 150 ) , & BG\_Level \geq 150\end{matrix}$$

According to some embodiments, the Risk Function may be constructed by the system (i.e., it's not hard coded). This is explained in greater details in FIG. 4 and its description.

Score Assignment:

According to some embodiments, the Score of Risk is calculated using the Risk Function, by accumulating the risk values of each data point in the dataset. This can be expressed in a general form:

$$Score = \frac{1}{minute} {{\int }_{0}}^{Total \_ Time} Risk\_Function ( Blood\_Glucose \_Level ( t ) ) dt$$

For discrete values with regular time intervals, this would be expressed as:

$$Score = \frac{Total\_Time}{minute} \cdot \frac{1}{N} {\sum }_{i = 1}^{N} Risk\_Function ( Blood\_Glucose \_Level [ i ] )$$

Where:

Risk_Function is the Risk Function, as described in this document. Units: dimensionless.

Blood_Glucose_Level returns the value of the blood glucose level in a certain time (for the continuous form), or at a certain position in the vector of blood glucose levels from the given dataset (for the discrete form). Units: [mg/dl] or [mmol/l].

N is the number of values in the dataset.

Total_Time is the time span that the dataset represents. Units: [minute].

Score is the Score of Risk. Units: dimensionless.

It should be noted that according to some embodiments the Score of Risk is dimensionless by design.

According to some embodiments, the time span that the dataset represents is not the time of the last value minus the time of the first value, but rather:

Total_Time=N·Time_Interval

Where:

Time_Interval is the regular time interval of the dataset.

This is because each value in the dataset represents the value of the blood glucose level over the time span of a single time interval. Units: [minute].

Several embodiments of the present disclosure:

Time of Day: In some Contexts of Risk, the time-of-day is considered relevant to the resulting Score of Risk. For example, some diet programs incorporate the theory that eating carbohydrates after a certain time (e.g., 19:00) is more likely to be harmful (whether strictly relating to weight loss purposes, or relating to other health issues).

The Score of Risk can be adjusted to reflect these time related effects, for example, by multiplying the blood glucose levels with an appropriate factor that depends on the time-of-day of the associated data points:

$$ToD\_Factor ( t ) = \{ \begin{matrix}1.0 , & t \leq 19 \\ 1 + \frac{( t - 19 )}{10} , & 19 > t > 20 \\ 1.1 , & t \geq 20\end{matrix} \\ Score = \frac{1}{minute} \int Risk\_Function ( Blood\_Glucose \_Level ( t ) \cdot ToD\_Factor ( t ) ) dt$$

Factoring the blood glucose level by a value greater than 1.0 when a risk value is assigned, reflects the proposition that a level that is considered less harmful for most of the day, should be considered as more harmful after evening (e.g., 140 [mg/dL] would be effectively evaluated as 154 [mg/dL] after 20:00).

Continuous Exposure Time:

Some exposure effects become more severe when the exposure time is continuous. An intuitive example for the importance of continuous exposure would be “putting a hand over a candle flame”. If done 30 separate times, each for 1 second, the resulting damage should be minimal. But if done 1 time, for a duration of 30 seconds, the damage can be expected to be a whole lot worse.

In some situations, the amount of continuous time of exposure to a harmful toxin should be factored. A straight forward implementation of this concept would start by setting a threshold, TH_{c}, which in this case was chosen to be:

TH_{c}=TH₀=150_{[mg/dL]}=8.3_{[mmol/l]}

During the calculation of the Score of Risk, a counter is incremented in each integration step that the blood glucose level is grater or equal to the threshold, and otherwise the counter is nullified. This counter effectively tracks the amount of minutes of continuous high exposure.

$$counter\_t• = \{ \begin{matrix}0 , & BG\_Level < {TH}_{c} \\ counter\_t ⊐ + Dt , & BG\_Level \geq {TH}_{c}\end{matrix}$$

The effect is designed to increase until it is capped at 120 continuous minutes:

$$f\_continuous = 1 + \frac{min ( counter\_t• , 120 )}{120}$$

Estimation and Utilization of Insulin Levels:

Insulin is the prominent hormone that regulates blood glucose levels. In diabetics this significance is expressed by the need to inject insulin in order to compensate for insulin deficiency. Insulin's importance can also be asserted by its incorporation, essentially, in every mathematical model of blood glucose dynamics.

Beyond its important for blood glucose regulation, insulin gains a lot of attention as a suspect for being the preamble of a main pathway for the development of insulin resistance. Moreover, there is mounting evidence for a strong correlation between high insulin levels and unhealthy metabolic symptoms such as weight gain, and fat storage.

This gives rise to a Context of Risk in which the Score of Risk reflects the risks relating to high insulin levels in a selected time span.

When using a simulation to estimate blood glucose levels from a Meal Plan, the insulin levels are already calculated as part of the existing model, so there are no extra steps needed to obtain an estimation of the insulin levels.

Another approach is using measured values of blood glucose levels (e.g., from a CGM) to estimate the corresponding insulin levels. In practice this would need some extra steps, and this approach may be less accurate (no food intake via Meal Plan).

When the Context of Risk is “risk of weight gain”, “risk of development of insulin resistance”, or other risks that are strongly associated with high insulin levels, the insulin estimation from the existing simulation can be used to calculate a relevant Score of Risk.

Some Contexts of Risk may utilize both blood glucose levels and insulin levels when calculating a Score of Risk. This synthesis could be achieved as a trivial adaptation and/or combination of implementations that were previously described.

In a same manner, other vitals may influence the calculated Score of Risk (e.g., heart rate, respiration rate, body temperature and perspiration level).

Score Presentation:

When presenting the Score of Risk, there are some aspects that are relevant in order to make it useful and coherent to the user.

Linear Scale and Logarithmic Scale:

The Risk of Score may be in a linear scale or in a logarithmic scale.

The advantage of the linear scale is that the Score of Risk would be additive, i.e., the Score of Risk assigned to an entire week would be equal to the sum of the Scores of Risk assigned to each day in that week.

The advantage of the logarithmic scale is that a large range of values can be presented in a compact manner that is easier to grasp.

Day and Month Views:

According to some embodiments, the main time scopes that are practical are “a day” and “a month”.

When the time scope is of a day, then the Score of Risk is calculated normally, based on the dataset of blood glucose/insulin levels for that day, and it may be presented next to other relevant information for that day.

In some embodiments, the “day time scope” is considered as the standard time scope for score assignment. By limiting the score assignment to a constant time span, the score can be presented in a logarithmic scale, as the downside regarding not being additive is mostly not relevant. If the time scope is not constant, it may be better to use a linear scale, as to have a coherent behavior over the different time scopes.

When the time scope is of a month, then each day within the month is assigned with a Score of Risk. This may be presented in a calendar style view that is populated with the Scores of Risk in the corresponding dates within the calendar (as opposed to calculating one Score of Risk for the entire time span of that month).

Convey Severity:

The higher the Score of Risk, the more risk for harm in the relevant Context of Risk. In situations that the Score of Risk is over a chosen threshold, the system may alert the user.

According to some embodiments, the system selects a threshold that is half of the expected upper range of the Score of Risk:

$${TH}_{warning} = \frac{upper\_score \_range}{2} = \frac{10}{2} = 5$$

When a Score of Risk is presented, if it is grater or equal to TH_{warning}, then the Score of Risk may be written in bold, and may have a highlighted background.

In some embodiments, if a Score of Risk that is already displayed while having a value below TH_{warning} gets updated with a value greater or equal to TH_{warning}, then an emphasizing animation is used to grab the user's attention to this.

Presenting with Other Metrics:

In some embodiments, the Score of Risk is presented with other metrics.

For example, when creating or viewing a Meal Plan in a specific date, other measures can be expected to be presented, such as:
Calories consumed that day.
Nutrients consumed that day (carbs, fats and protein).

When reviewing blood glucose levels, examples for relevant measures that may also be presented are:
Minimum/maximum/average blood glucose level.
Estimated A1C.

One problem dealt by the present disclosure is how to provide an effective monitoring of blood glucose levels. If diabetes is left untreated, it can cause many complications. Acute complications can include diabetic ketoacidosis, hyperosmolar hyperglycemic state, or death. Serious long-term complications include cardiovascular disease, stroke, chronic kidney disease, foot ulcers, and damage to the eyes. As the condition progresses, determining what is an effective and relevant representation of risk should also change.

One solution is to provide a monitoring system that is tailored to the current health status of the user. The risk analysis is based on a Context of Risk that is relevant to the user's current condition, and as the condition evolves, the Context of Risk is updated accordingly.

One other technical solution is providing a system for risk analysis and management. This system utilizes measurements of blood glucose/insulin that originate from the monitored user, which reflect the user's health condition and behavior. This system provides feedback to the user in the form of risk-based scores, of which its values are directly dependent on the measured levels of glucose and/or insulin. This feedback mechanism may assist in managing the user's risk exposure.

One other technical problem dealt by the present disclosure is the absence of meaningful and actionable feedback for blood glucose levels. Users of devices such as CGM are able to review their blood glucose levels in the form of graphs over time, embellished only by some basic statistical measures. The health implications are practically intangible, and are left to the users' subjective interpretations of the graphs. This is addressed by the simple yet coherent and informative Score of Risk, that while encapsulates the accumulated health risks, is presented in a form of a single value that is scaled to be in a range that is both easy to grasp, and yet clearly conveys the effects of small changes. This should support the user in taking proactive actions, as the effects of even small changes in the Meal Plan can provide the user with feedback that should help with enforcing positive habits. The Score of Risk provides:
A single value for an entire day.
Clear meaning (directly represents a measure of risk).
Consistent and coherent measure that can be compared: a higher Score of Risk over time (e.g., a year) indicates of a deterioration in the health condition of the user.
A reliable and credible measure that eliminates guesswork, along with its associated cognitive and mental loads (e.g., for when reviewing or making choices).
Facilitating presentation of trends of the Score of Risk, providing a way to review over large time scales, by creating a graph where each day has a scalar score value.
Real time response to changes in user's behavior. An immediate feedback when making chooses, assists with internalizing the expected impact of different actions.

One other technical solution is utilizing stored data of the user's previous blood glucose levels, analyzing the data to assess a Context of Risk that is relevant to the user, and modifying accordingly the way that the Score of Risk is calculated by updating the Risk Function. Such a solution provides:
A Context of Risk (and thus also the resulting Score of Risk) that stays relevant to the current condition of the user.
A range of the resulting Score of Risk that is neither too high nor too low; Somewhat like when adjusting the TV volume so that it's neither too loud nor too quiet.

One other problem dealt by the present disclosure is how to provide guidance in everyday food choices. This is addressed by estimating the expected influence of a food item that the user considers to eat over the user's blood glucose/insulin levels. This is done by utilizing a Glucose-Insulin Simulation to estimate future values of glucose and insulin and assigning a Score of Risk. This gives the user a tool to evaluate the ramifications of different choices and provides a basis for a methodological way for prioritization. Such a solution provides:
Real time personally tailored feedback. Such a feedback may influence the decisions of the user; Guide the user to make informed and cost effective decisions, such that negative health effects could be minimized, while not having to over-restrict oneself.
A relief to users from the stress that arises from the uncertainty regarding the adverse effects of what and when they eat. In other words, eliminate the burden that stems from having the responsibility for make choices with overwhelming amount of uncertainties.

### THE BRIEF DESCRIPTION OF THE SEVERAL VIEWS OF THE DRAWINGS

The present disclosed subject matter will be understood and appreciated more fully by following the detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1A shows a block diagram of a system for monitoring blood glucose levels, in accordance with some exemplary embodiments of the subject matter;

FIG. 1B depicts the system for monitoring blood glucose levels, in accordance with some exemplary embodiments of the subject matter;

FIG. 2 shows a flowchart diagram of a method for performing glucose-insulin simulation, in accordance with some exemplary embodiments of the subject matter;

FIG. 3 shows a flowchart diagram of a method for calculating Score of Risk, in accordance with some exemplary embodiments of the subject matter;

FIG. 4 shows a flowchart diagram of a method for constructing a Risk Function, in accordance with some exemplary embodiments of the subject matter;

FIG. 5 shows a flowchart diagram of a method for assigning Context of Risk, in accordance with some exemplary embodiments of the subject matter;

FIG. 6 shows a flowchart diagram of a method for on the fly planning, in accordance with some exemplary embodiments of the subject matter;

FIG. 7 shows a flowchart diagram of a method for generating a new Meal Plan Template, in accordance with some exemplary embodiments of the subject matter;

FIG. 8 shows a flowchart diagram of a method for generating a list of Meal Plan Template variations, in accordance with some exemplary embodiments of the subject matter;

FIG. 9 shows a flowchart diagram of a method for analyzing and informing the user regarding the risk inscribed within the data from an entire previous month, in accordance with some exemplary embodiments of the subject matter;

FIG. 10 shows a block diagram of a compartment-model of glucose-insulin simulation;

FIG. 11 shows a graph of natural history of type 2 diabetes;

FIG. 12 shows a visual composition of two graphs, illustrating how the risk is assessed from the user's blood glucose levels; in accordance with some exemplary embodiments of the subject matter;

FIG. 13 shows a visual composition of two graphs, illustrating the concept of a more effective glucose toxicity risk assessment at night time; in accordance with some exemplary embodiments of the subject matter;

FIG. 14 shows a calendar style summary, with daily Scores of Risk presented in their corresponding dates within the calendar; in accordance with some exemplary embodiments of the subject matter;

FIG. 15 shows a screen shot of a Meal Plan Template; in accordance with some exemplary embodiments of the subject matter;

FIG. 16 shows a table with values for deriving Contexts of Risk or for deriving Risk Functions; in accordance with some exemplary embodiments of the subject matter;

FIG. 17 shows a screenshot of the graphic user interface used for adding a food item on the fly, in accordance with some exemplary embodiments of the subject matter; and

FIG. 18 shows screen shots of the graphic user interface in various stages of the process of selecting a Meal Plan Template, in accordance with some exemplary embodiments of the subject matter.

### DETAILED DESCRIPTION

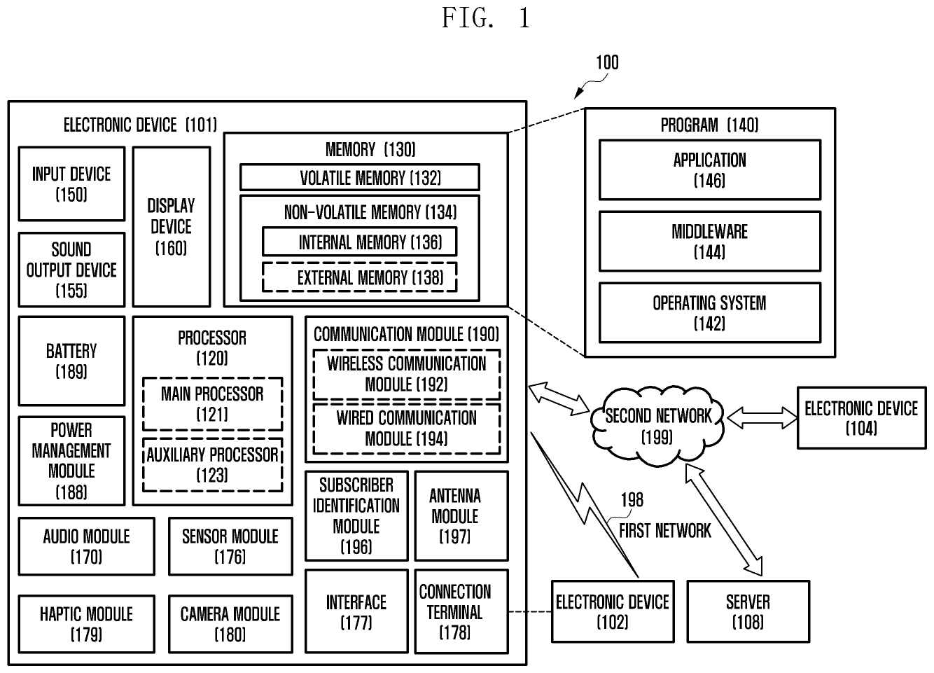

FIG. 1A shows a block diagram of a system for monitoring blood glucose levels, in accordance with some exemplary embodiments of the subject matter.

System 100 includes a sensor 101, an intermediate sub system 102, a data aggregate service 103 and a processing unit 104.

The sensor 101 may be any measuring device that senses the glucose level and/or the insulin level in the blood or in the interstitial fluid. Example of such a sensor is the FreeStyle Libre Sensor by Abbott.

The intermediate subsystem 102 is configured for transmitting the sensor data to a data storage 1031 or to an interface of a data storage. The intermediate subsystem may include a mobile device or a wearable device or a computing device or a combination of mobile, wearable, and computing devices. An example of a mobile device is the iPhone. An example of a wearable device is the Apple Watch. An example of a computing device is the MacBook Pro 13-inch, Late 2009.

The intermediate subsystem may include a reader 1021 which is configured for reading the output of the sensor and a communication device 1022 which is configured for communicating with the data storage or with an interface of a data storage. An example of a reader is the FreeStyle Libre Reader by Abbott. An example of a communication device is a laptop computer such as the MacBook Pro 13-inch, Late 2009.

The data aggregate service 103 is configured for storing and retrieving data.

The data aggregate service 103 includes a data storage 1031 and may include an online data aggregator 1032. The data storage 1031 is configured for storing data received from the sensor. The online data aggregator 1032 is a service that is configured for interfacing with the data storage. Example of such service is Google Fit.

The processing unit 104 includes a processing device 1041, an interface device 1042, a glucose-insulin simulation module 1044, a Score of Risk calculation module 1045, a Context of Risk determination module 1046, a Risk Function generation module 1047, and a generation of refined Meal Plan Template module 1048.

The processing device 1041 is configured for processing glucose-insulin simulation module 1044, a Score of Risk calculation module 1045, a Context of Risk determination module 1046, a Risk Function generation module 1047, and a generation of refined Meal Plan Template module 1048.

The interface device 1042 is configured for implementing a user interface.

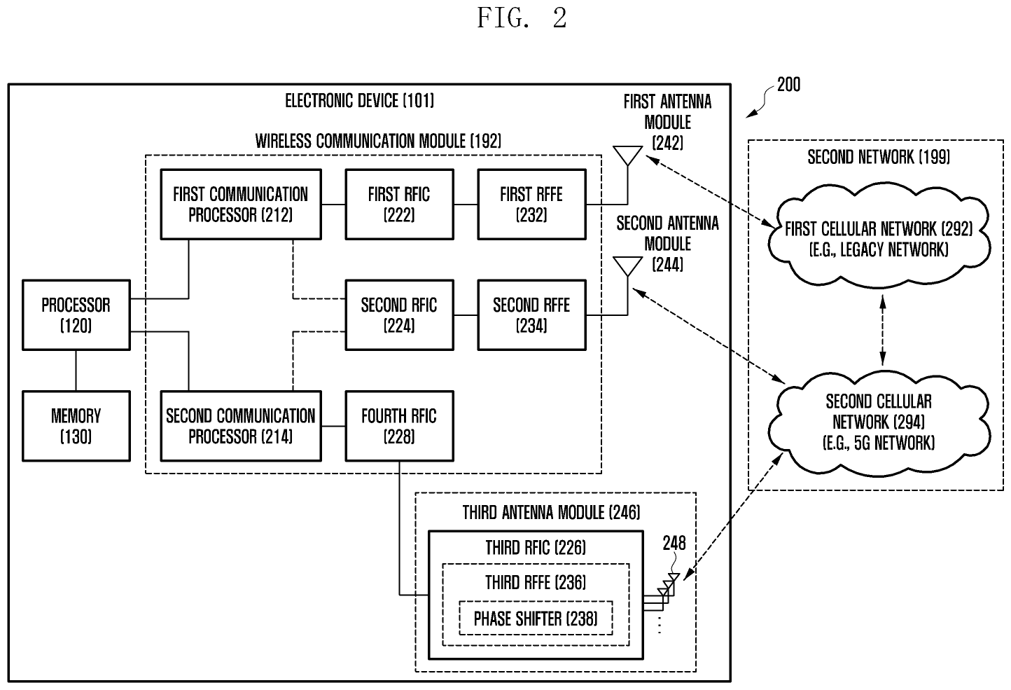

The glucose-insulin simulation module 1044 is configured for simulating the concentration of glucose and insulin in the plasma, according to the Meal Plan. The output of the process is a dataset for the time span that is simulated. The process of the glucose-insulin simulation module 1044 is explained in greater detail in FIG. 2.

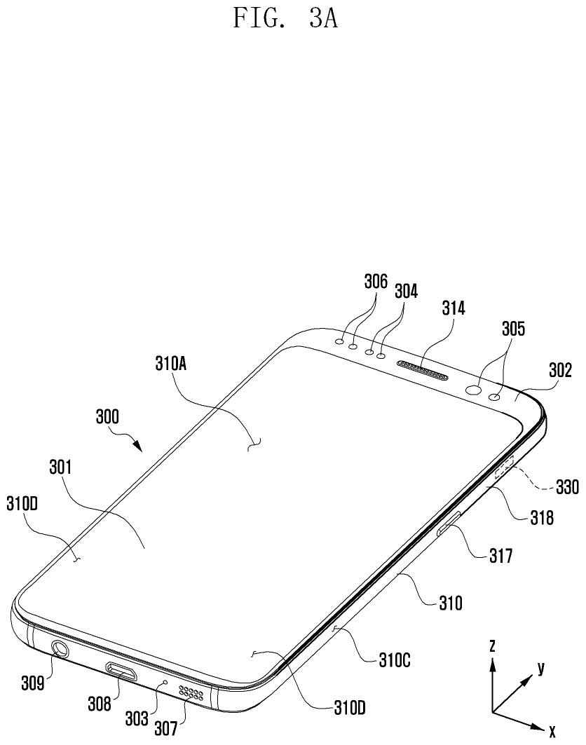

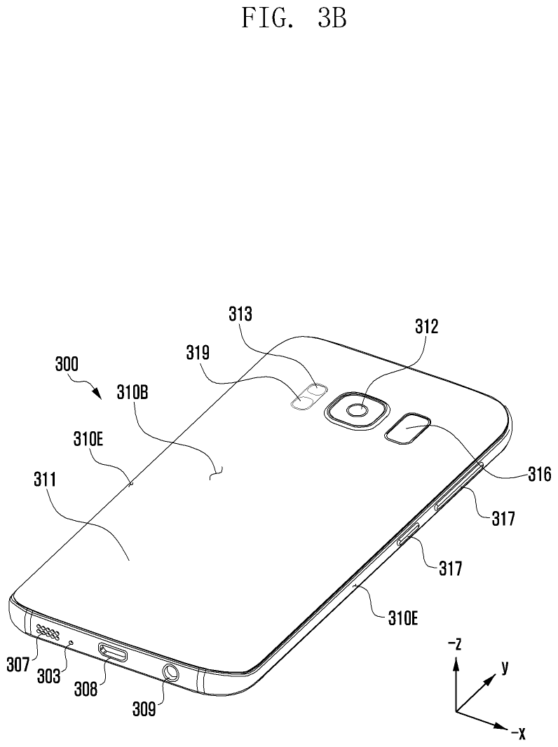

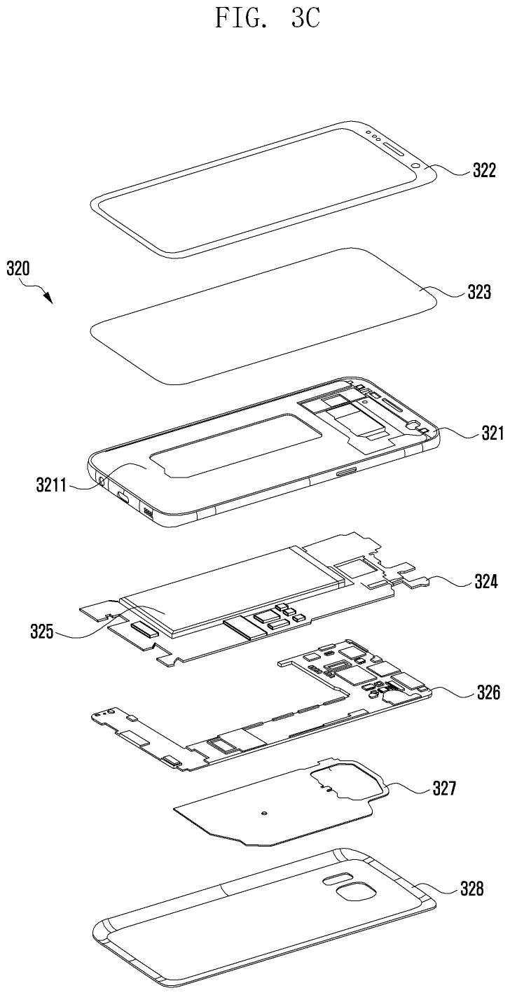

The Score of Risk calculation module 1045 is configured for calculating the Score of Risk. According to some embodiments, the Score of Risk represents the accumulated risk that is associated with the toxicity of blood glucose/insulin levels over the time span of the dataset and as relevant for the selected Context of Risk. The process of calculating the Score of Risk may be performed upon user request or when activating the system. The process of the Score of Risk calculation module 1045 is explained in greater detail in FIG. 3.

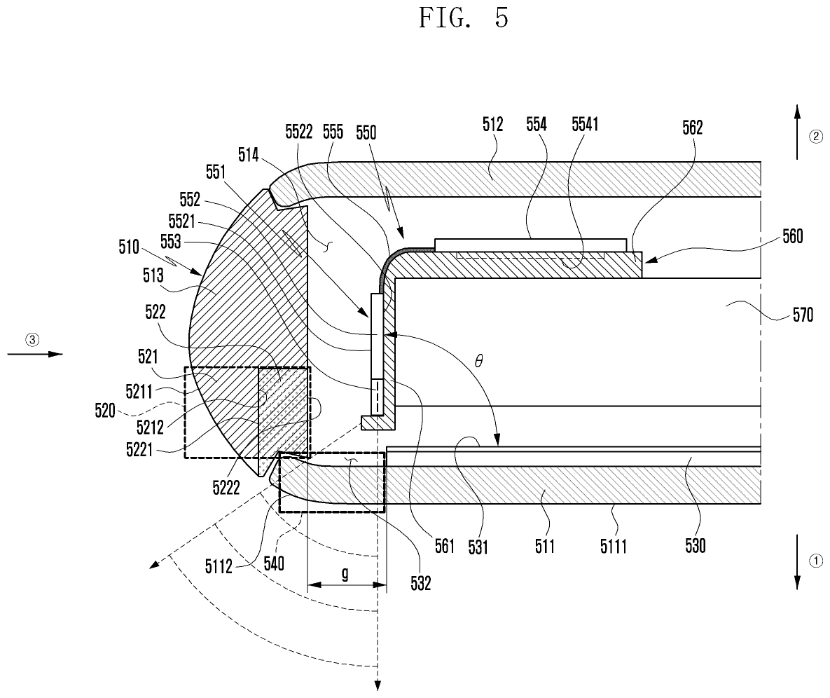

The Context of Risk determination module 1046 is configured for determining the Context of Risk. According to some embodiments, the system periodically determines the Context of Risk in accordance with the history of blood glucose/insulin levels of the user. The process of the Context of Risk determination module 1046 is explained in greater detail in FIG. 5.

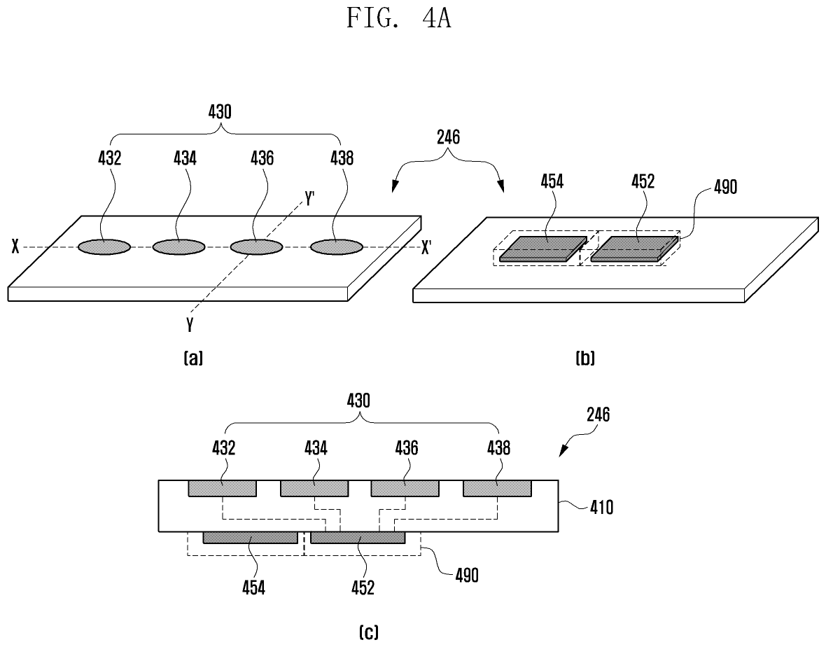

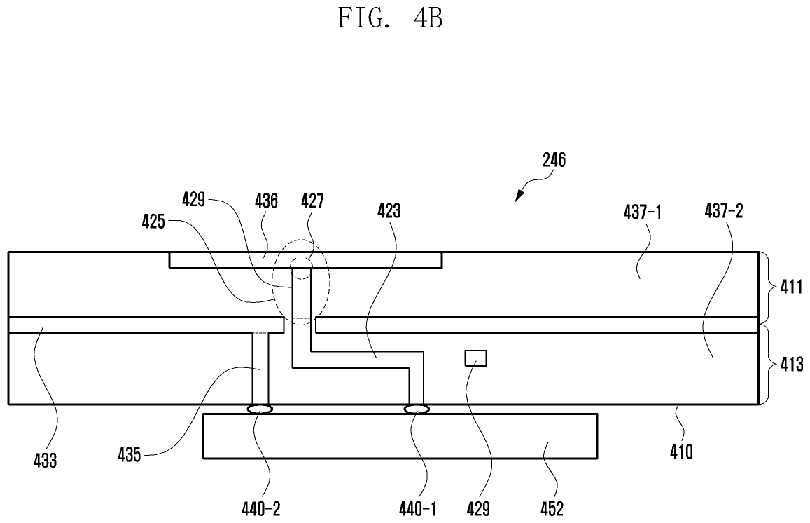

The Risk Function generation module 1047 is configured for generating the Risk Function. According to some embodiments, the Risk Function is constructed in accordance with the Context of Risk. The process of the Risk Function generation module 1047 is explained in greater detail in FIG. 4.

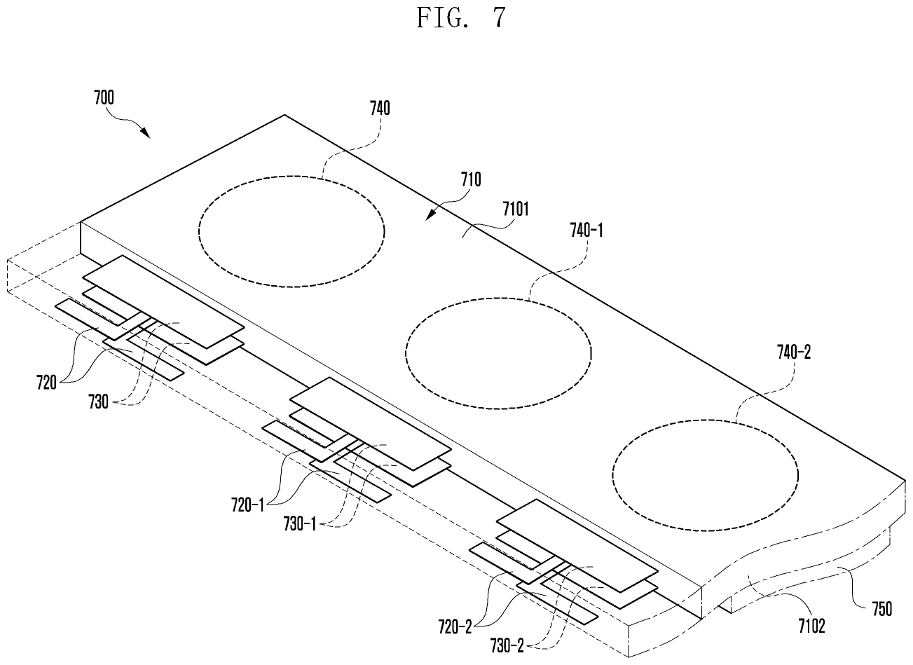

The generation of refined Meal Plan Template module 1048 is configured for generating a new Meal Plan Template or for refining an existing Meal Plan Template. The process of generation of refined Meal Plan Template module 1048 is explained in greater detail in FIG. 7.

FIG. 1B depicts the system for monitoring blood glucose levels, in accordance with some exemplary embodiments of the subject matter. System 100 includes the sensor 101 attached to a person 111, a reader 1021 as the intermediate device, the communication device 1022 the data aggregate service 103, and the processing unit 104.

FIG. 2 shows a flowchart diagram of a method for performing glucose-insulin simulation, in accordance with some exemplary embodiments of the subject matter. According to some embodiments, the glucose-insulin simulation process estimates concentration levels of glucose and of insulin within the plasma compartment and within the interstitial fluid compartment—according to the Meal Plan. The output of the process is a dataset for the time span that is simulated.

At block 201 the Meal Plan is received from the data repository.

At block 203 the glucose-insulin simulation parameters are adapted according to the profile of the user. Examples for such parameters are: the basal plasma glucose concentration, and the diffusion rate parameters between the different model-compartments.

At block 205 the counters of the process are initiated. In particular, the simulation-time is initiated.

Blocks 207, 209, 212, 215 and 217 include the process for generating a data point for the current processed interval time.

At block 207, time is propagated, and counters are incremented for the current iteration.

At block 209 the system queries the data repository for retrieving Meal Plan food-items associated with the time interval of the current iteration. If such food-items exist, then in block 213 the carbohydrate values of the food-items are added and the process continues to block 212.

Otherwise, operation continues to block 212.

At block 212 the simulation state is propagated. The propagation may be performed, for example, according to “Meal Simulation Model of the Glucose-Insulin System” by C. Dalla Man et al, as depicted in FIG. 10.

At block 215 the glucose concentration within the plasma compartment and the insulin concentration within the interstitial fluid compartment are stored in the dataset.

At block 217 the system checks if the current processed simulation-time is the last time interval to be processed. If the answer is positive, then the operation continues to block 219; otherwise, the operation returns to block 207.

At block 219 the subsystem returns the dataset.

FIG. 3 shows a flowchart diagram of a method for calculating the Score of Risk, in accordance with some exemplary embodiments of the subject matter. According to some embodiments, the Score of Risk represents the accumulated risk that is associated with the toxicity of the blood glucose/insulin levels over the time span of the dataset, and as relevant for the selected Context of Risk. The process of calculating the Score of Risk may be performed upon user request, or upon activation of the system.

At block 300 the dataset is retrieved from the data repository.

At block 301 the system checks if the Risk Function has to be replaced. The Risk Function has to be replaced if the Context of Risk has been changed, or if there is no preexisting Risk Function. If the Risk Function has to be initiated or replaced, then at block 302 the system constructs a new Risk Function, and the operation continues to block 303. The process of constructing a new Risk Function is explained in greater detail in FIG. 4.

Otherwise (i.e., no need to construct a new Risk Function), the operation continues to block 303.

At block 303 the system initializes values for the process of summing risk values of all time intervals within the time-span of the retrieved dataset.

Blocks 304, 305 and 306 calculate the accumulated risk value by summing up the risk values from each time interval in the evaluated time span.

Blocks 304, 305 and 306 are repeated for all of the encompassed time intervals.

At block 304 the system increments counters.

At block 305 the system adds the risk value of the current time interval to a summation variable. The risk value of the current time interval is based on the current glucose level and/or the current insulin level.

The risk that is attributed to glucose is given by applying the Risk Function over the current blood glucose level. In some embodiments the blood glucose level is first multiplied by the Time-of-Day Factor to reflect the higher sensitivity to glucose at night time as expressed by some dieticians (illustrated in FIG. 3). In some embodiments, a counter keeps track of how many continuous time intervals have their associated blood glucose level over a certain threshold. This counter is used to increase the “effective risk value”, and thus reflect the higher effective exposure that occurs when the exposure is continuous. This can be justified due to local diffusion time through cell-membranes.

The risk value that is attributed to insulin is given by applying the insulin Risk Function over the current insulin level within the Interstitial Fluid compartment.

At block 306 the system checks if this is the last interval time. If the answer is positive, then the system proceeds to block 307, otherwise, the system returns to block 304.

At block 307 the system calculates the total time and the Score of Risk. The total time is calculated by multiplying the time interval duration with the number of iterations. The Score of Risk is calculated by dividing the summation variable by the number of iterations, and then multiplying the result by the total time's number of minutes. In embodiments where the scale of the Score of Risk is logarithmic, a log function is applied over the later product to create the proper Score of Risk.

At block 308 the subsystem returns the Score of Risk.

FIG. 4 shows a flowchart diagram of a method for constructing a Risk Function, in accordance with some exemplary embodiments of the subject matter. According to some embodiments, the Risk Function is constructed in accordance with the Context of Risk.

At block 400 the system checks if there is a relevant Context of Risk.

If the Context of Risk does not exist or needs to be updated, then at block 420 the system presents to the user a list of Contexts of Risk from which the user is requested to select one. The system may also generate a new Context of Risk. The process of generating the Context of Risk is explained in greater detail in FIG. 5. The operation continues to block 405.

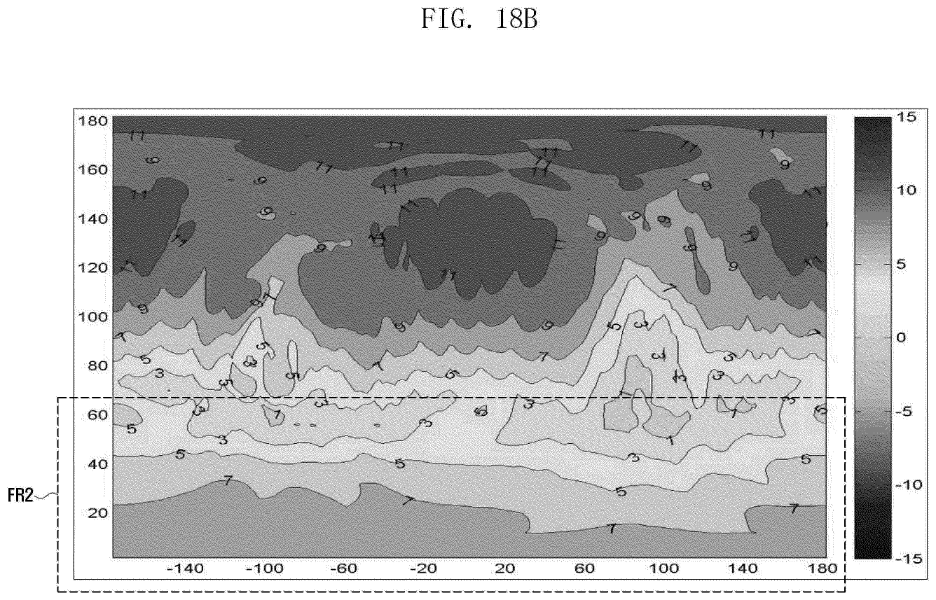

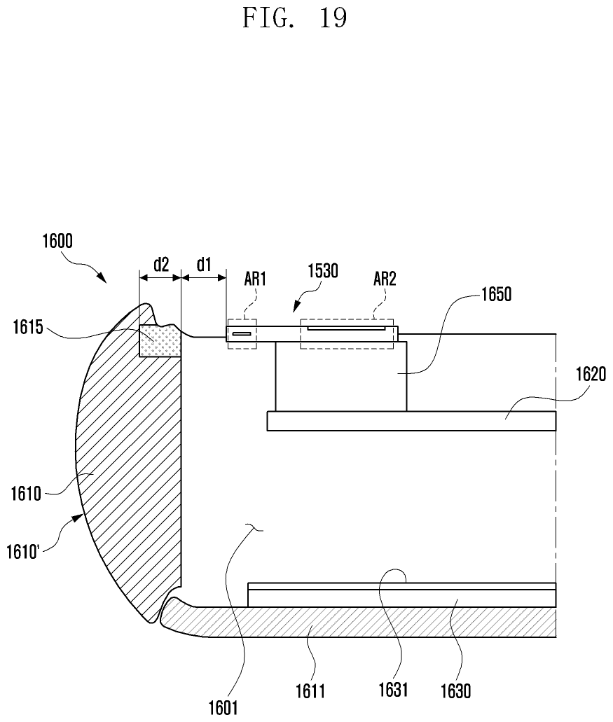

At block 405 the construction values of the Risk Functions which are associated with the selected Context of Risk are retrieved from the Context-Data-Table (depicted in FIG. 16). The construction values contain parameters such as the threshold values of the Risk Functions (TH1 and TH_Insulin) and the expected high range values of the Risk Functions (BG_H and Ins_H).

At block 410 the slopes of the Risk Functions are derived. The Risk Functions have a zero value below their respective threshold values, and have linearly increasing values above those thresholds—in the form of a line that stems from the X axis. A straight line can be defined by two points: the first point is the line's origination point from the threshold value, which is (TH1, 0). The second point defines the high range of the expected glucose/insulin levels for users for which the current Context of Risk is relevant. In some embodiments, the Risk Function is scaled, such that the weight of risk is set to be 20 for the blood glucose level that is equal to the high end of the expected range of blood glucose levels. Thus, the second point is (BG_H, 20). In this example the slope is:

$${F}_{1} = \frac{{y}_{2} - {y}_{1}}{{x}_{2} - {x}_{1}} = \frac{20}{BG\_H - {TH}_{1}}$$

For the Insulin Risk Function the slope is derived in the same manner:

$${F}_{2} = \frac{{y}_{2} - {y}_{1}}{{x}_{2} - {x}_{1}} = \frac{20}{Ins\_H - TH\_Insulin}$$

At block 415 the subsystem returns the Risk Functions that are used to generate a Score of Risk in accordance with the blood glucose levels and with the insulin levels.

FIG. 5 shows a flowchart diagram of a method for assigning Context of Risk, in accordance with some exemplary embodiments of the subject matter. According to some embodiments, the system periodically reevaluates the Context of Risk for the user, according to the user's history of blood glucose levels and/or insulin levels.

At block 500 the system queries the history of the user's blood glucose levels. In one example, the system queries from a data repository a dataset that includes the user's blood glucose levels for the last month.

At block 501 the system checks the return values for the query from the data repository. If a dataset was retrieved, then the system continues to block 503. Otherwise, the system continues to block 502.

At block 503 BG_Basal is calculated. BG_Basal is a value that indicates the lower range of the blood glucose levels from the queried period. In one example the value is the mean of the minimal value of the average value of the blood glucose levels:

$$BG\_Basal = \frac{\text{Average(Dataset)} + Min \text{(Dataset)}}{2}$$

Operation continues to block 504.

At Block 502 BG_Basal is set based on the user's Fasting Plasma Glucose (FPG) as the indicator for the expected lower range of the blood glucose levels.

At block 504 the system initializes counters.

Blocks 505,506 and 507 disclose the process of deriving the Context of Risk from the Context-Data-Table, and for deriving the corresponding Risk Function. The Context-Data-Table is explained in greater detail in FIG. 16.

The process searches for the first TH1 value that is sufficiently greater than BG_Basal.

At block 505 the counter of the rows in the table is incremented.

At block 506 the threshold value from the List-TH1 column is retrieved.

At block 507 BG_Basal is compared to the retrieved threshold.

If BG_Basal is greater or equal to the retrieved threshold (0.9·TH1), then the operation continues block 508. Otherwise, the operation continues to block 509.

At block 508 the system checks if the counter of rows is equal to number of rows (last row has been checked). If they are equal, then the operation continues to block 510. Otherwise, operation returns to block 505.

Blocks 510 and 512 disclose the process of determining an adaptive Context of Risk. The values for deriving the Risk Function are calculated based on the value of BG_Basal.

At block 510 the Context of Risk is identified as an adaptive context, and a distinct value is assigned to the Context_Index to express an adaptive Context of Risk. In one example, the Context_Index for the adaptive Context of Risk is equal to 0.

At block 512 the system assigns values to TH1, BG_H, TH_Insulin and Ins_H. These values are used for constructing the Risk Function, as elaborated in the description for block 410. In one example, the insulin related values are set to zero, and the glucose related values are calculated based on BG_Basal:

TH1=1.2*BG_Basal,TH_Insulin=0

BG_H=2*BG_Basal,Ins_H=0

Block 509 and 511 disclose the process of determining the Context of Risk, and the values for the Risk Function according to the Context-Data-Table.

At block 509 the value of the current counter of rows is determined as the Context_Index, which represents a specific Context of Risk.

At block 511 the system assigns values to TH1, BG_H, TH_Insulin, and Ins_H from the current row (i.e., Context_Index) in the Context-Data-Table. These values are used for constructing the Risk Function.

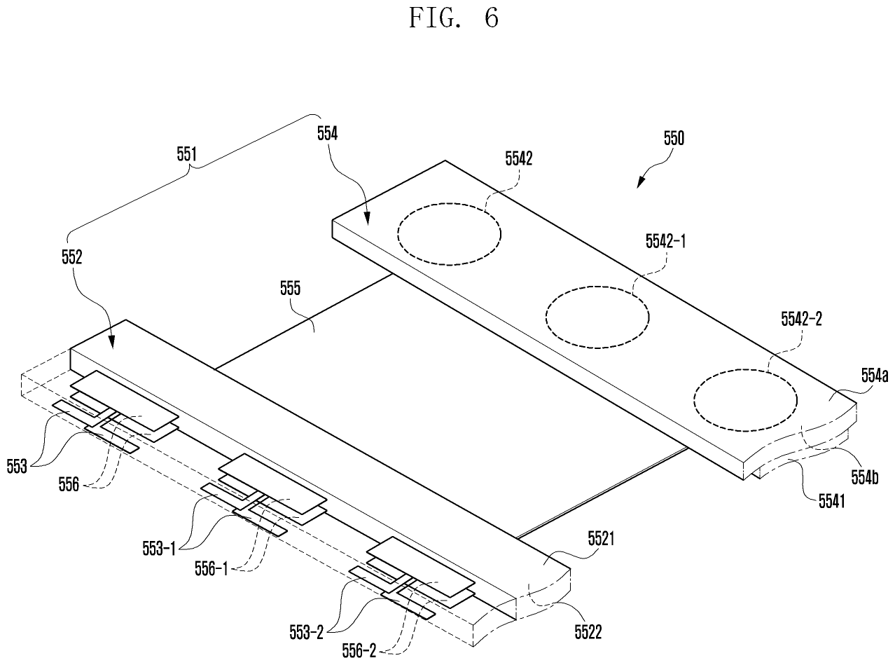

FIG. 6 shows a flowchart diagram of a method for on the fly planning, in accordance with some exemplary embodiments of the subject matter. According to some embodiments, the system provides the user the ability to change a Meal Plan, and using the continuously updating Score of Risk to determine in real time which actions are preferable in terms of minimizing health risks.

At block 600 the system receives a request from the user to add a new food item to the Meal Plan. The request includes date and time, the identification of the food item and the quantity of the food item.

At block 605 the system changes the Meal Plan in accordance with the user's request. The system adds the new item to the diary (i.e., the processing device 1041).

At block 610 the system operates the glucose-insulin simulation. The method for operating the glucose-insulin simulation is explained in greater detail in FIG. 2. The glucose-insulin simulation estimates the levels of blood glucose and of insulin according to the modified Meal Plan.

At block 615 the system assigns a Score of Risk according to the simulated blood glucose level and according to the Context of Risk that is currently selected for the user. The process of calculating the Score of Risk is explained in greater detail in FIG. 3.

At block 620 the system checks if the Score of Risk is above a threshold. If the Score of Risk is above a threshold, then the system proceeds to block 625. Otherwise, the system continues to block 630.

At block 625 the system alerts the user about the high Score of Risk. The notification is according to the Context of Risk. The notification may be implemented by the presentation of alert-messages, and may also provide tips for lowering the risk. The notification may be sent to the user's processing unit 104, and may be presented on its interface device 1042. The operation continues to block 630.

At block 630 the system presents the new Score of Risk for the modified Meal Plan with the additional food item.

It should be noted that this process is also applicable for changing the amount of an existing food item in the Meal Plan, or for removing a food item from the Meal Plan, or for changing the time in which a food item in the Meal Plan is consumed.

FIG. 7 shows a flowchart diagram of a method for optimizing a Meal Plan Template, in accordance with some exemplary embodiments of the subject matter. According to some embodiments, the Meal Plan Template optimization provides the user an option for planning meals while keeping the Score of Risk as low as possible.

At block 700 the user selects a Meal Plan Template from a list of Meal Plan Templates. The system receives the selected Meal Plan Template.

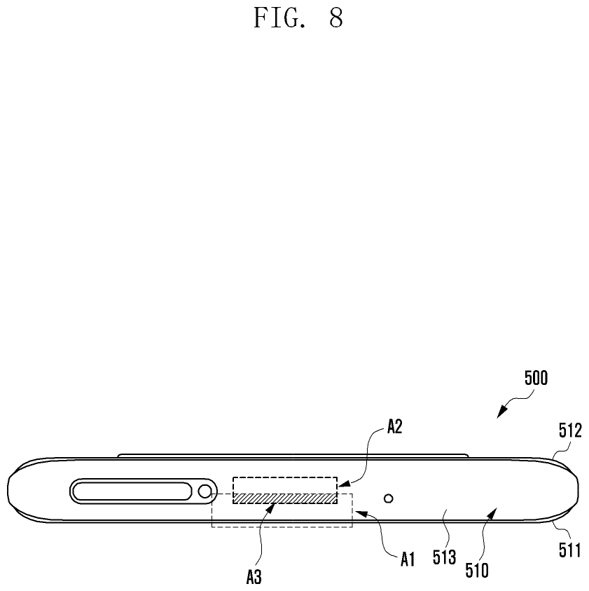

At block 705 the system generates a list of Meal Plan Template variations. The process of generating the list is explained in greater detail in FIG. 8.

Blocks 710,715,720,725 and 730 disclose the method for assigning a Score of Risk to each Meal Plan Template variation.

At block 710 one of variations is selected ordinally from the list.

At block 715 a glucose-insulin simulation is performed, generating a dataset associated with the current Meal Plan Template variation.

At block 720 a Score of Risk is assigned to the dataset of the current variation.

At block 725 the system associates (in the data repository) the Score of Risk with the current Meal Plan Template variation.

At block 730 the system checks if all of the Meal Plan Template variations have been assigned with a Score of Risk. If the answer is positive, then operation returns to block 710. Otherwise, the operation continues to block 735.

At block 735 the system selects from the data repository the Meal Plan Template variation with the lowest Score of Risk.

At block 740 the selected Meal Plan Template variation is added to the list of Meal Plan Templates that are available to the user.

FIG. 8 shows a flowchart diagram of a method for generating a list of Meal Plan Template variations, in accordance with some exemplary embodiments of the subject matter.

At block 800 the system receives the selected Meal Plan Template.

At block 805 the food counter is initialized. The food counter indicates the food item that is currently modified (set to 0, where 1 corresponds to the first item).

Blocks 810, 815, 820, 825, 830, 835, 840, 845 and 850 disclose the process of generating Meal Plan Template variations.

At block 810 the food counter for the current food item is incremented.

At block 815 the system retrieves the index and the time associated with of the current food item.

At block 820 the system generates a time list. The time list includes optional variations of time for the current food item.

Blocks 825, 830, 835, 840 and 845 disclose the process of generating a variation of a Meal Plan Template in accordance with the optional variations of time for the current food item.

At block 825 the system selects ordinally a time variation from the time list.

At block 830 the system generates a new Meal Plan Template which is a copy of the selected Meal Plan Template.

At block 835 the system changes the time of the current food item in the new Meal Plan Template to the selected time variation from the time list.

At block 840 the new variation of the Meal Plan Template is saved in the list of Meal Plan Template variations.

At block 845 the system checks if the selected time variation is the last time variation in the time list (for the current food item).

If this is the last item, then the operation continues to block 850, otherwise, the operation returns to block 825.

At block 850 the system checks if all food items were processed.

If all food items have been processed, then at block 855 the system returns the list of all meal plan template variations, otherwise, the operation returns to block 810.

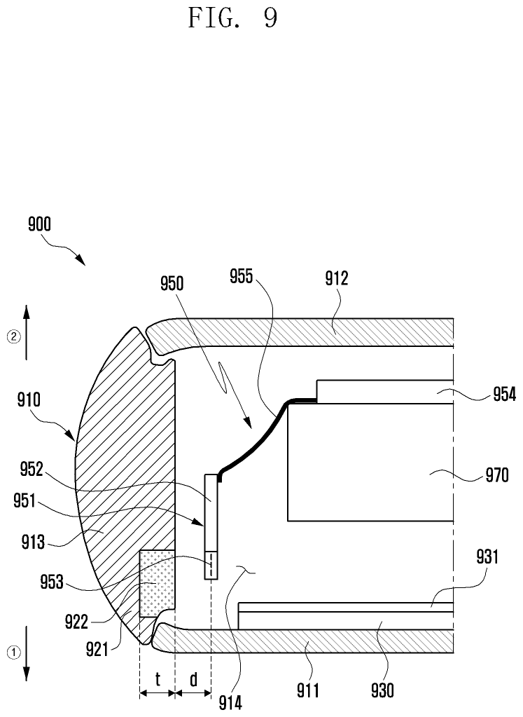

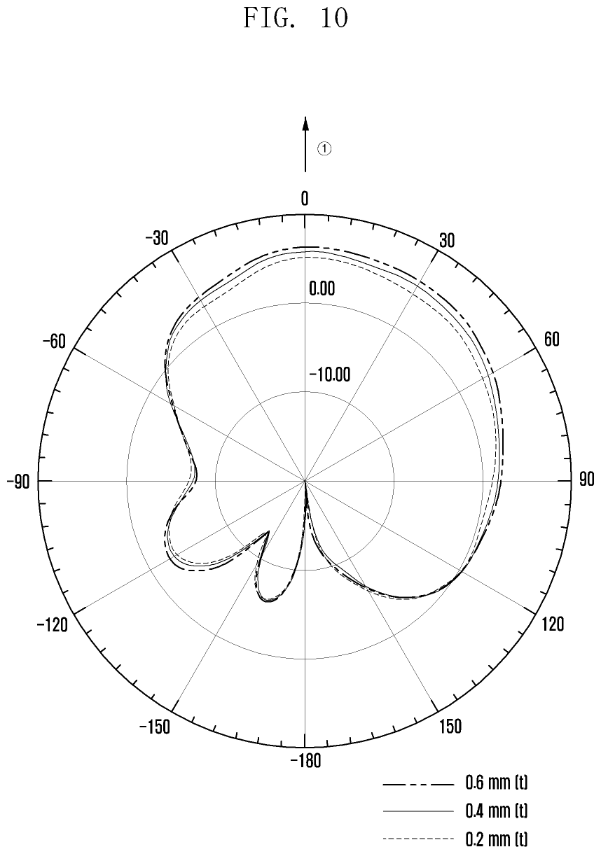

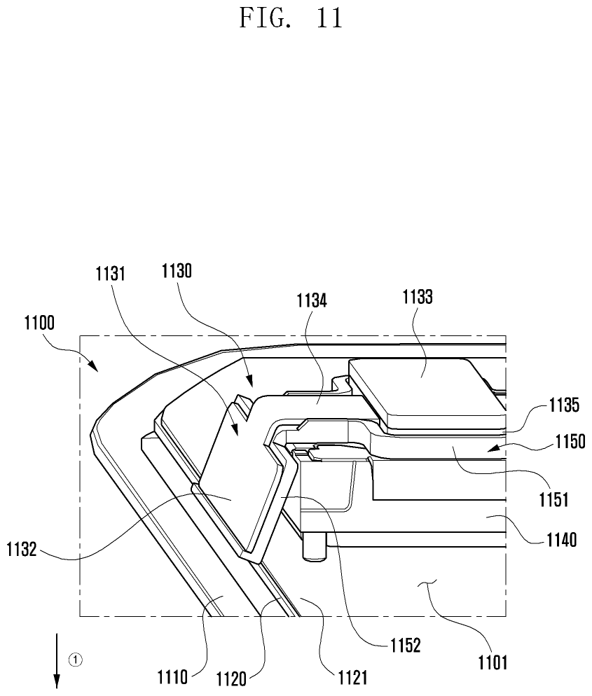

FIG. 9 shows a flowchart diagram of a method for analyzing the history of the user, in accordance with some exemplary embodiments of the subject matter.

At block 900 the user requests to review the history on the basis of assigned values of Score of Risk. For example: last month's period.

At block 905 the system, in response to the user's request, queries the user's blood glucose levels for last month period, and receives the dataset for the last month.

At block 910 the system separates the month's dataset into a list of daily datasets.

Blocks 915, 920, 925 and 930 disclose the process of presenting the history of the Score of Risk.

At block 915 the system selects ordinally a dataset from the list of daily datasets.

At block 920 the system assigns a Score of Risk for the selected dataset. The process of calculating the Score of Risk is explained in greater detail in FIG. 3.

At block 925 the system presents the Score of Risk within a calendar style view. The system highlights the score if it is above a threshold.

At block 930 the system checks if this was the last dataset in the list of daily datasets. If it is the last one, then the system proceeds to block 935; otherwise, the system returns to block 915.

At block 935 the system checks in how many days the Score of Risk exceeds a certain threshold.

If the number of days exceeds another threshold, then the operation continues to block 940, otherwise, the operation continues to block 945.

At block 940 the system notifies the user about the severity of the results.

At block 945 the system presents the calendar overview to the user.

FIG. 10 is a block diagram of a prior art of a compartment-model of blood glucose simulation. The figure discloses a Diagram by MathWorks® for SimBiology implementation of: Meal Simulation Model of the Glucose-Insulin System. C. Dalla Man, R. A. Rizza, and C. Cobelli. IEEE Transactions on Biomedical Engineering (2007) 54(10), 1740-1749. When referring in several flowchart blocks to a specific compartments (i.e., Dose, Plasma and Interstitial Fluid) the reference is made in accordance with this diagram which presents the different compartments of the model, and which is fully detailed in the mentioned article.

FIG. 11 shows a graph of natural history of type 2 diabetes.

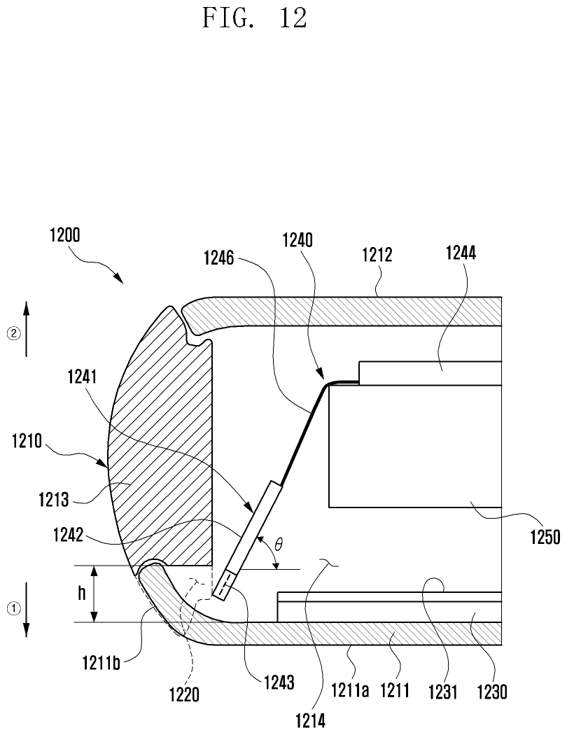

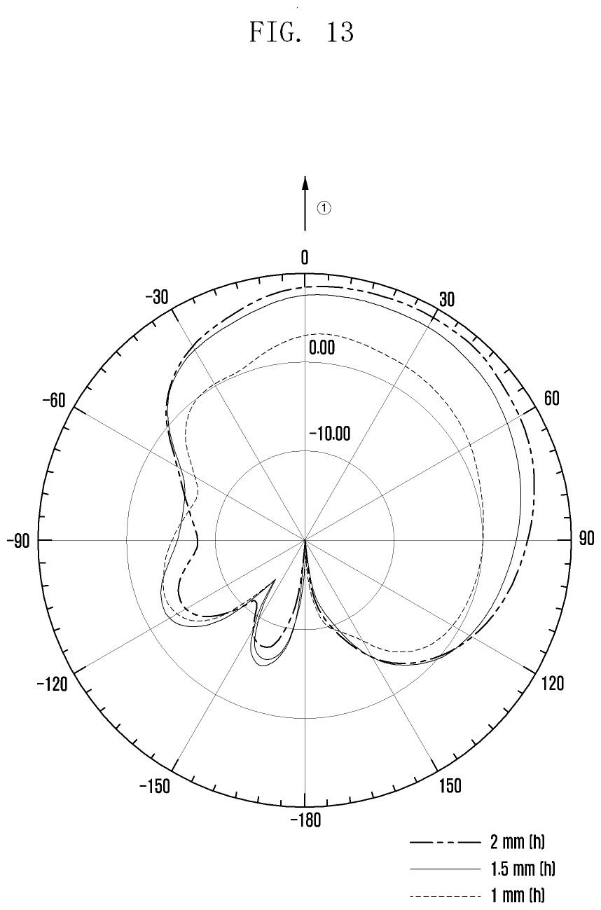

FIG. 12 shows a visual composition of two graphs, illustrating how the risk is assessed from the user's blood glucose levels. Graph 1201 shows a graphical representation of the user's blood glucose levels over a day's period. Graph 1202 shows the Risk Function (transposed for illustrative purposes), in accordance with some exemplary embodiments of the subject matter. According to some embodiments, the Risk Function is based on the Threshold model. The Threshold mode has a concentration value (its namesake threshold value) for which it is known (or assumed) that for concentrations bellow it, the risk is zero. For simplicity, the behavior above the threshold-concentration is assumed to increase linearly, such that it emerges from the threshold-concentration (the corresponding line crosses the horizontal concentration axis at the threshold-concentration). The figure discloses a Risk Function in which the threshold-concentration value is 150.

Graph 1201 shows the blood glucose level values of a user throughout the day. The x axis represents the hours of the day. The Y axis represents the glucose levels.

Graph 1202 shows the Risk Function (transposed). The Y axis represents the blood glucose levels (which is the function's argument in this transposed representation) and the X axis represents the risk weights assigned to the corresponding blood glucose levels. Risk value isn't the same as risk weight; it is the total/compound value from all relevant risk sources (may be equal to the risk weight in single factor risk models).

FIG. 13 shows a visual composition of two graphs, illustrating the concept of higher effective glucose-toxicity at night time; in accordance with some exemplary embodiments of the subject matter.

Graph 1301 shows the blood glucose level values of a user throughout the day. The x axis represents the hours of the day. The Y axis represents the blood glucose level values.

Graph 1302 shows the Time of Day factor. According to some embodiments, the risk due to blood glucose levels changes throughout the day. In the exemplary graph 1302 the risk is higher after 20:00, with proper transition values between 19:00 to 20:00.

Graph 1301 contains graph plots 13011 and 13012, and illustrates how the Risk Function is affected by the Time of Day factor. Graph plot 13012 represent the original values. Graph plot 13011 represent the “effective values”, with the Time of Day factor.

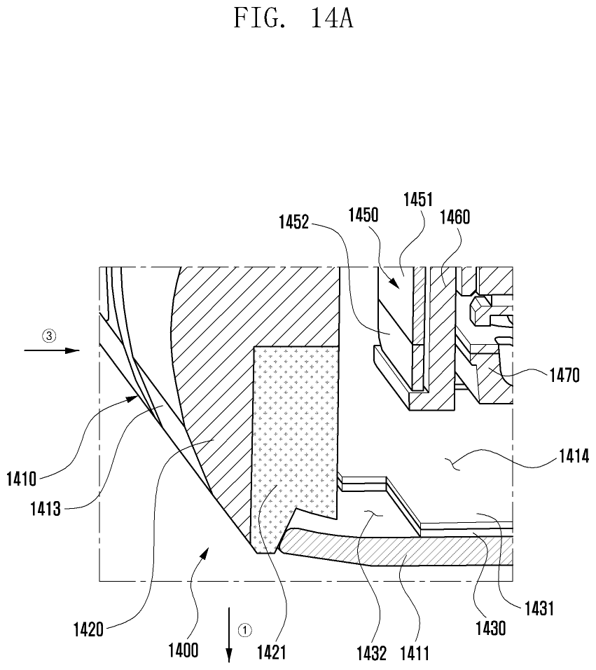

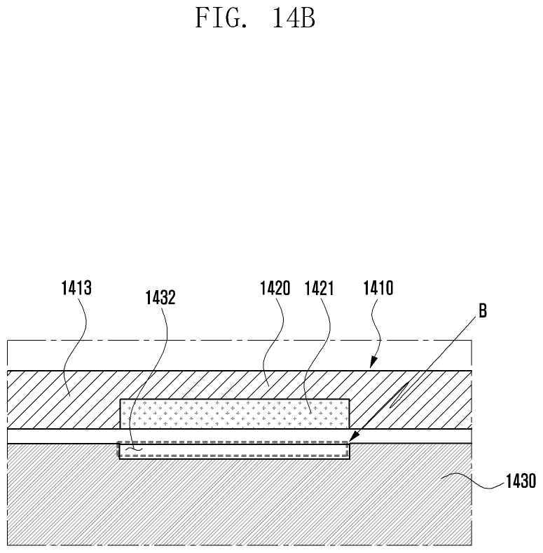

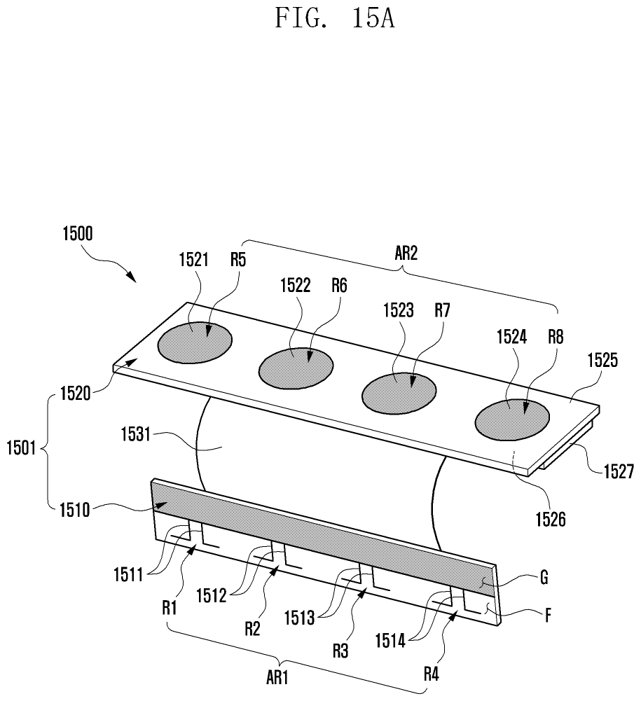

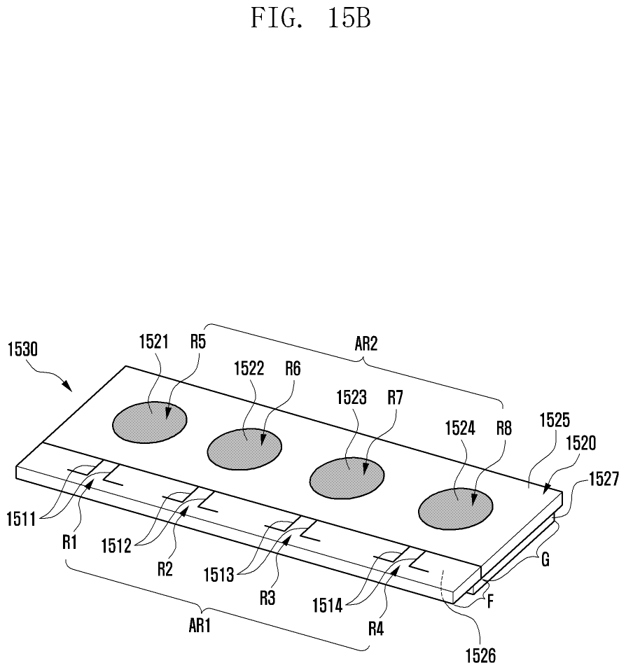

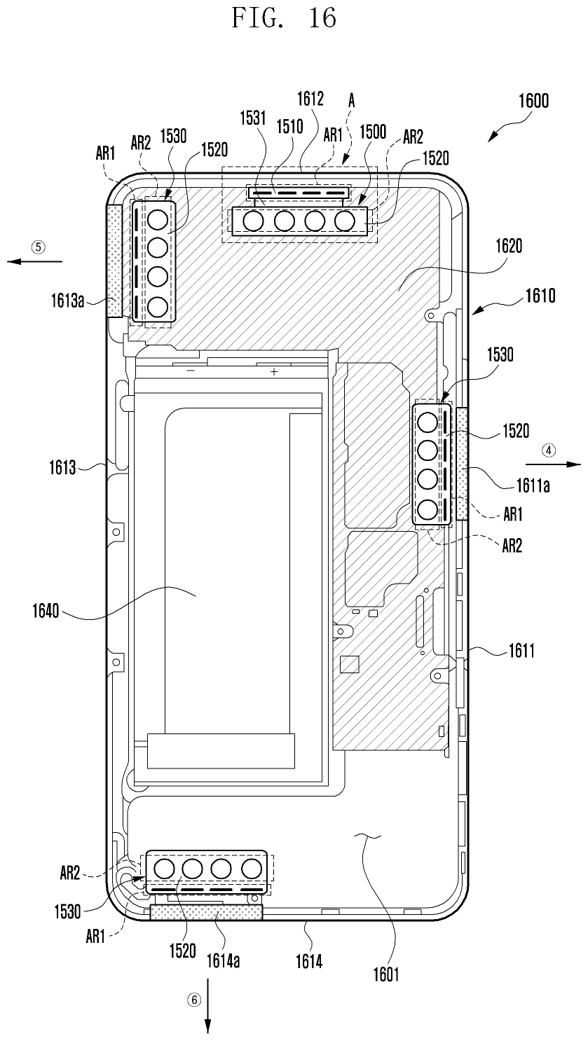

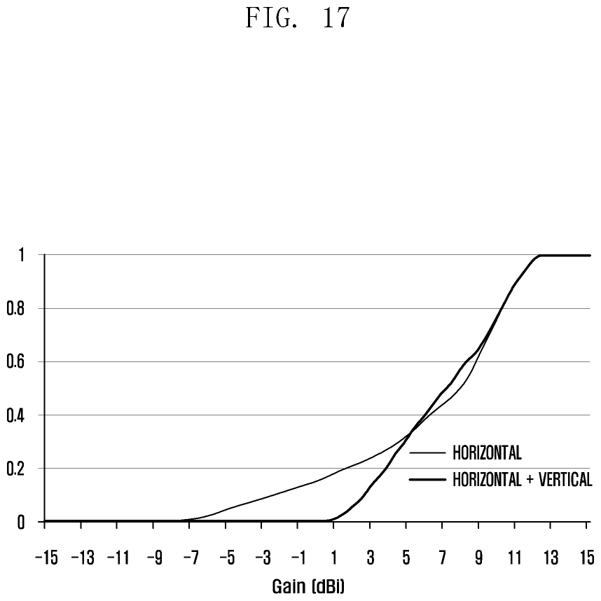

FIG. 14 shows a summary calendar view; Summary calendar view 1400 is created from the method for reviewing past month's daily Scores of Risk. The method of assigning Scores of Risk in this manner is explained in greater detail in FIG. 9. The summary calendar 1400 displays the Scores of Risk for each day. For example, the Score of Risk 1402 is within a normal range, while the Score of Risk 1401 is highlighted since it is above a threshold.

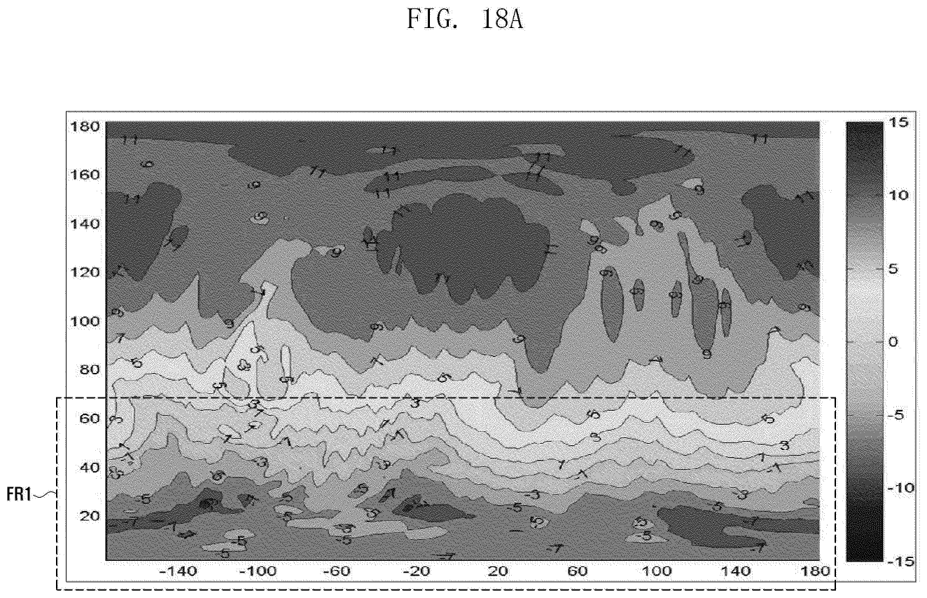

FIG. 15 shows a screen shot of a Meal Plan Template; in accordance with some exemplary embodiments of the subject matter. Meal Plan Template 1500 includes a Score of Risk 1502, a graph of the blood glucose levels throughout the day 1501, and list of food items 1503.

FIG. 16 shows a table depicting a Context-Data-Table that can be used for deriving the Context of Risk or for deriving the Risk Function, in accordance with some exemplary embodiments of the subject matter.

$$Risk\_Model ( BG\_Level ) = \{ \begin{matrix}0 , & BG\_Level < {TH}_{1} \\ {F}_{1} \cdot ( BG\_Level - {TH}_{1} ) , & BG\_Level \geq {TH}_{1}\end{matrix}$$

### Where:

BG_Level is the current blood glucose level. Units: [mg/dl] or [mmol/l].
TH₁ is the threshold-concentration as described above. Units: [mg/dl] or [mmol/l].
F₁ is the Factor that determines the slope, as described above. Units: [dl/mg] or [1/mmol].

F₁ is determined by checking the range of the scores associated with typical low and high blood glucose levels for the user that requires the assessment of the risk, as related to the current Context of Risk.

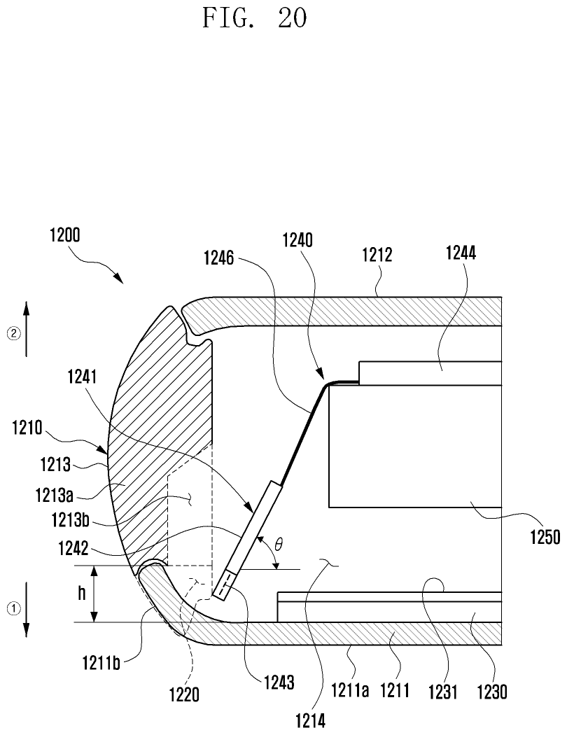

FIG. 17 shows a screenshot of the process of adding a food item on the fly, in accordance with some exemplary embodiments of the subject matter. Screen 1700 displays a slider 1701 for adjusting the amount of the food item and a slider 1702 for allowing the user to adjust the time of day in which the food item is to be consumed.

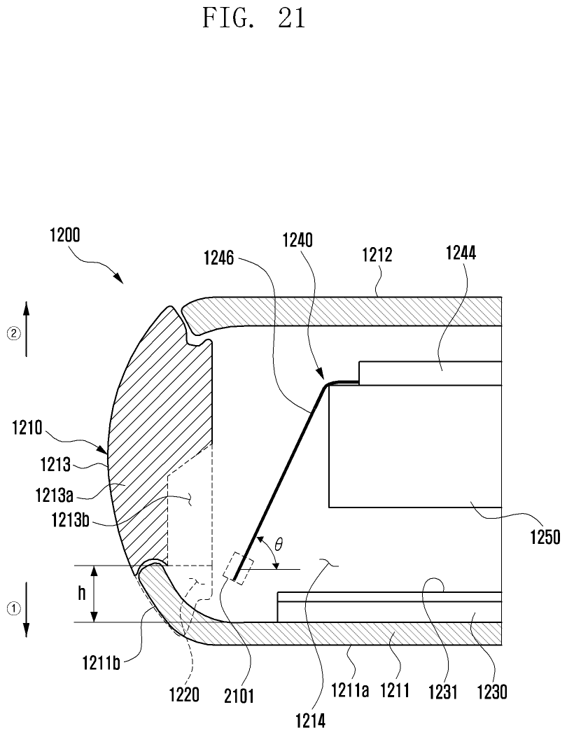

FIG. 18 depicts screenshots for the process of selecting a Meal Plan Template; in accordance with some exemplary embodiments of the subject matter.

1801 shows the planning calendar into which templates can be embedded.

1802 shows the available Meal Plan Templates.

1803 shows a detailed view of an exemplary Meal Plan Template.

The terminology used herein is for the purpose of describing only particular embodiments, and it is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It should be noted that, in some alternative implementations, the functions noted in the block of a figure may occur out of the order noted in the figures. For example, two blocks shown in succession may, in fact, be executed substantially concurrently, or the blocks may sometimes be executed in the reverse order, depending upon the functionality involved.

What is claimed is:

1. A method, the method comprises:
receiving from a sensor or from a processor a dataset of a plurality of data points; wherein each data point from said plurality of data points comprises a first value of a level of glucose or a second value of a level of insulin in a certain time interval within a time period, wherein said sensor sampling a user or wherein said processor simulating said dataset in accordance with behavior of a user;
calculating score of risk for toxicity over said time period, wherein said toxicity is associated with a level of insulin or a level of glucose in blood; wherein input of said calculating comprises said data set; and
if said score of risk exceeding a threshold then causing a presentation of said score of risk on a computing device or causing an initiation of an alert.

2. The method of claim 1, wherein said calculating utilizing a risk function, said risk function resulting in a plurality of risk-values, each associated with a data point from said plurality of data points, wherein each of said plurality of risk-values reflecting damage due to blood toxicity associated with said data point in said time interval, wherein said calculating further comprises performing integration of said plurality of risk-values over said time period.

3. The method of claim 2, wherein said risk function being derived from a context of risk, wherein said context of risk being a frame of reference for said risk function, said frame of reference defining toxicity range values for said risk function.

4. The method of claim 3, wherein said context of risk being selected in accordance with a history of blood glucose level of said user or in accordance with a history of insulin level of said user in a selected time period.

5. The method of claim 3, wherein said context of risk being calculated in accordance with a history of blood glucose level of said user or in accordance with a history of insulin level of said user in a selected time period.

6. The method of claim 1, further comprising counting number of time periods in which said score of risk exceeding said threshold and if said number of time periods exceeding a second threshold and causing the presentation of an alert via a computing device.

7. The method of claim 1, wherein said behavior is associated with a diet of said user.

8. The method of claim 1, wherein said method being implemented by one member of a group consisting of: a mobile device, a wearable device and a computing device.

9. A method, the method comprises:
receiving a meal plan of a user;
analyzing blood glucose level resulting from utilizing said meal plan or analyzing insulin level in blood resulting from utilizing said meal plan, said analyzing resulting in a dataset of data points, wherein each data point from said dataset comprises a first value of said blood glucose level of said user or a second value of said insulin level in blood of said user and a time interval corresponding with said first value or with said second value; and
generating a plurality of variations of said meal plan;
calculating score of risk for damage due to blood toxicity for each dataset resulted from each variation of said plurality of variations and selecting a meal plan associated with lowest score of risk.

10. The method of claim 9, further comprising recalculating said score of risk as a result of adding a new food item to said meal plan.

11. At least one non-transitory computer-readable storage medium having stored thereon instructions that, when executed by one or more processors, cause the one or more processors to perform operations comprising:
receiving from a sensor or from a processor a dataset of a plurality of data points, wherein each data point from said plurality of data points comprises a first value of a level of glucose or a second value of a level of insulin in a certain time interval within a time period, wherein said sensor sampling a user, or wherein said processor simulating said dataset in accordance with behavior of a user, calculating score of risk for blood toxicity over said time period, wherein said toxicity is associated with a level of insulin or a level of glucose in blood, wherein input of said calculating comprises said data set,
if said score of risk exceeding a threshold then causing a presentation of said score of risk on a computing device or causing an initiation of an alert.

12. The method of claim 1, wherein said behavior is associated with the diet of the user.
